(19) United States
(12) Patent Application Publication
(10) Pub. No.: US 2003/0147954 A1
(43) Pub. Date: Aug. 7, 2003

(54) Cyclosporin-containing sustained release pharmaceutical composition

(75) Inventors: Jeong Hwa Yang, Seongnam-si (KR); Ham Yong Park, Gangnam-gu (KR); Dong Woo Lim, Jungnang-gu (KR); Jung Ju Kim, Yongin-si (KR)

Correspondence Address: HESLIN ROTHENBERG FARLEY & MESITI PC, 5 COLUMBIA CIRCLE, ALBANY, NY, 12203, US

(73) Assignee: PACIFIC CORPORATION, Seoul (KR)

(21) Appl. No.: 10/356,752

(22) Filed: Jan. 30, 2003

(30) Foreign Application Priority Data
Feb. 1, 2002 (KR) 2002-5856

(51) Int. Cl. A61K 38/13; A61K 31/724; A61K 35/78; A61K 9/22

(52) U.S. Cl. 424/468; 514/11; 514/58; 424/750; 424/757; 424/727; 424/776

(57) ABSTRACT

A pharmaceutical composition formulated for sustained release is disclosed. In one embodiment, the pharmaceutical composition comprises cyclosporin and a release modifier encapsulated in a biodegradable polymer. In a preferred embodiment, the release modifier is selected from the group consisting of hydrophilic release modifiers, lipophilic release modifiers, and combinations thereof. Most preferably, the release modifier comprises at least one hydrophilic release modifier and at least one lipophilic release modifier.

### CLAIM FOR FOREIGN PRIORITY

This application claims foreign priority benefits from Korean Patent Application Number 2002-5856, which was filed Feb. 1, 2002. The entire content of the prior application is incorporated herein by reference.

### FIELD OF THE INVENTION

The present invention relates to cyclosporin-containing sustained release pharmaceutical compositions.

### BACKGROUND OF THE INVENTION

Until now, a main area of clinical research on cyclosporin has been in regard to its use as an immunosuppressive agent, particularly its administration to recipients of organ transplants, such as, for example, heart, lung, combined heart-lung, liver, kidney, pancreas, bone marrow, skin and corneal transplants and specifically allogeneic organ transplants. In this field, the utilization of cyclosporin has achieved remarkable success.

Another use of cyclosporin has been in the treatment of various autoimmune diseases and inflammatory conditions, particularly those induced by etiologic factors, and an autoimmune component in arthritis and rheumatic diseases, has been emphasized. Many reports and in vitro results in animal models and in clinical trials have been frequently disclosed in the literature. Specific auto-immune diseases for which cyclosporin therapy has been proposed or applied, include, but are not limited to, autoimmune hemolytic diseases (including, for example, hemolytic anemia, aplastic anemia, normocytic anemia and idiopathic thrombocytopenia), systemic lupus erythematosus, polychondritis, scleroderma, Wegener's granulomatosis, dermatomyositis, chronic active hepatitis, myasthenia gravis, psoriasis, Stevens-Johnson syndrome, idiopathic sprue, autoimmune inflammatory bowel diseases (including, for example, ulcerative colitis and Crohn's disease), endocrine opthalmopathy, Graves' disease, sarcoidosis, multiple sclerosis, primary biliary cirrhosis, juvenile diabetes mellitus (genuine diabetes type I), uveitis (anterior and posterior), keratoconjunctivitis sicca, vernal keratoconjunctivitis, interstitial pulmonary fibrosis, psoriatic arthritis and glomerulonephritis (with or without nephrotic syndrome, e.g., including idiopathic nephrotic syndrome or minimal lesion nephritic syndrome).

Additional research with cyclosporin has recognized its potential applicability as an antiparasitic, particularly as an anti-protozoal agent, and it has also been suggested for use in the treatment of malaria, coccidiomycosis and schistosomiasis. More recently, cyclosporin has been used as an agent for reversing or eliminating antineoplastic-resistance of tumors and the like.

While cyclosporin is the most widely used of the various immunosuppressive agents, it does have one particular disadvantage: it suffers from a very low level of oral bioavailability. Upon oral administration, 10 to 27% of the total absorbed amount is subjected to the first pass effect in liver. The distribution half-life for cyclosporin is 0.7 to 1.7 hours and its elimination half-life is 6.2 to 23.9 hours. Such pharmacokinetic parameters of cyclosporin vary significantly from patient to patient, depending on the secretion level of bile acid, the overall physical condition of the patient, as well as the type of organ transplant the patient has undergone. Other disadvantages of cyclosporin include adverse renal effects such as a reduction of glomerular filtration rate, an increase of proximal renal tubular reabsoprtion, and the like. It has been reported that about 30% of patients taking cyclosporin formulations will develop some degree of nephrotoxicity due to the high levels of cyclosporin in the blood. Thus, patients undergoing therapy with cyclosporin must be subjected to periodic therapeutic drug blood level monitoring.

Due to the many very specific characteristics of cyclosporin administration, i.e., very low solubility, low bioavailability, widely varying absorption rates among patients, high dosage requirements and a narrow therapeutic index, especially in combination with the already unstable physical condition of the patient being treated, it is very difficult to establish an optimum drug dosage regimen that can ensure survival of the patient, through maintenance of an effective drug blood concentration, while avoiding potentially dangerous adverse effects and organ rejection.

Due to its poor and variable bioavailability, it is necessary to monitor the patient's blood concentration on a daily basis and adjust the dose of cyclosporin accordingly, in order to achieve and maintain a desired blood concentration. Currently, the initial dose of cyclosporin is determined on the basis of data obtained from analysis of the patient's blood concentration patterns observed following administration of the drug prior to the actual transplant operation. With the rapid advances in development of organ transplant medical technology, the frequency and types of transplants will steadily increase, creating a dire need for immunosuppressive agents such as cyclosporin that can be administered easily and be therapeutically effective. Current cyclosporin treatment is enormously expensive, due to the medical expense for the initial blood concentration analysis to determine a starting daily does for each individual patient, as well as the frequent, and often daily, therapeutic drug monitoring that must occur.

Therefore, there is a significant need for a cyclosporin pharmaceutical formulation that not only has high oral bioavailability, but that is not affected by individual patient physiological differences, and can maintain a constant blood concentration in each patient.

While there have been attempts to enhance the bioavailability of cyclosporin, and while improved formulations have been developed, such attempts have mainly focused on means to solubilize cyclosporin. Typical examples include the use of liposomes, microspheres, mixed solvent systems consisting of general vegetable oils and surfactants, the formation of powdery compositions using adsorption complexes, inclusion complexes, solid dispersions, etc., and the like. In general, cyclosporin formulations have been for oral administration.

One important attempt to improve the bioavailability of cyclosporin is described in U.S. Pat. No. 5,342,625. This reference discloses a microemulsion pre-concentrate comprising a three-phase system: (1) a hydrophilic phase component; (2) a lipophilic phase component; and (3) a surfactant component. The formulation also includes alcohol as an essential component and provides an oil-in-water microemulsion having an average particle size of less than about 100 nm upon dilution with water. This greatly increased surface area provided improved cyclosporin bioavailability as compared to conventional dosage forms.

In vivo comparisons of the microemulsion formulation (Composition I from the '625 patent) with conventional formulations based on ethanol and oil (e.g., Composition X disclosed in U.S. Pat. No. 4,388,307), were conducted on healthy volunteers and the results reported in the '625 patent. Composition I records a bioavailability level of 149.0% (±48), as compared with Composition X (for which bioavailability achieved is set as 100%). Although the average area under the curve (“AUC”) value of Composition I is 40% higher than that of Composition X, its deviation of 20% is too large for practical use in a medicinal preparation.

U.S. Pat. No. 5,641,745 discloses microspheres comprising cyclosporin entrapped in a biodegradable polymer, which are capable of releasing more than 80% of the entrapped cyclosporin within an 8 hours, thereby maximizing absorption of cyclosporin in the small intestine. This technology thus provides cyclosporin preparations with improved bioavailability, by maximizing the release of cyclosporin entrapped in poly(lactide) in the upper small intestine, where cyclosporin is predominantly absorbed. Upon study of this formulation, however, the phenomenon that more than 80% of the drug is released within 8 hours of administration is considered to be due to the initial burst of drug (typical for microsphere-type preparations), rather than release regulation by the biodegradable polymer. It has also been suggested that the release amount varies according to the poly(lactide) content in the polymer. Furthermore, it is believed that the solubility of cyclosporin depends on its form, i.e., amorphous and crystalline, which varies according to the type of polymer, and not due to the controlled release of cyclosporin by the biodegradable polymer. In practical use, no additional drug release after the 8 hour initial release was observed during the remaining test period.

Therefore, while this formulation is suitable for oral preparations which should complete release in a targeted organ (upper small intestine), it is not suitable for controlled release preparations that are required to continuously release drug over an extended period of time. Moreover, it is hard to expect long-term drug delivery by oral administration. Low and non-uniform oral absorption levels of cyclosporin is due to individual patient differences, and it is therefore anticipated that administration of cyclosporin by other routes may overcome many of the drug's difficulties.

While there are commercially available injectable cyclosporin preparations, these include solubilizers such as polyoxyethylated castor oil derivatives, which may induce hypersensitivity reactions, and the use of such preparations is limited to patients who cannot undergo oral therapy.

In an attempt to address this problem, U.S. Pat. No. 5,527,537 discloses a pharmaceutical composition containing cyclosporin for intravenous administration, which does not contain polyoxyethylated castor oil derivatives. However, due to the fact that treatment with cyclosporin routinely occurs daily for a very long period of time, IV administration is not an ideal substitute for oral administration.

Recently, results have been reported for a biodegradable microsphere preparation including poly(lactide) or poly(lactide-co-glycolide) that can continuously release cyclosporin over an extended period of time. The researchers reported that microspheres containing cyclosporin showed rapid release of drug in vitro at the early stage, followed by sustained-release, with the maximum being 50% for 4 weeks (Int'l. J. Pharmaceut., 99:263-273, 1993). Even with the regulation of particle size (a typical method for regulation of a drug release pattern), only the initial release burst was increased, and an increase in the release rate was not seen. This is believed to be due to the fact that release is restricted by the interaction between the cyclosporin and the poly(lactide-co-glycolide) at the later release stages. The phenomenon that in vitro release of drug almost never occurs at the later release stages is frequently observed not only with hydrophobic drugs, but also with hydrophilic protein drugs. Considering the biodegradable characteristics of polymers, it is difficult to reproduce the in vitro release pattern in an in vivo test situation. In any case, the maximum release rate of 50% for 4 weeks recognizes that there remains a need for a formulation that provides additional drug release.

Fairly recent research has demonstrated the potential for increasing the in vitro release of cyclosporin by adding various fatty acid esters to the formulation. (Urata, T. et al., “Modification of release rates of cyclosporin A from polyl (L-lactic acid) microspheres by fatty acid esters and in vivo evaluation of the microspheres,” J. Controlled Release, 58:133-141, 1999). The study reveals that lipophilic cyclosporin was considered to be mainly solubilized in the fatty acid ester and the fatty acid ester was dispersed in poly(lactide), and that the solubilized drug was subsequently released through water channels formed by the fatty acid ester. All of the fatty acid esters employed in the study are liquids at room temperature, except for ethyl stearate. However, since ethyl stearate has a melting point of 33 to 35° C., it also becomes a liquid at 37° C., which is the temperature of the human body as well as the temperature of in-vitro release tests.

Thus, as only cyclosporin dissolved in the liquid phase can be released over time, a desired increase of release rate can be attained when the content of the fatty acid ester based on the total weight of preparation is 30% or more, such that the cyclosporin is sufficiently dissolved. The microspheres have been prepared using poly(lactide) or polylactide co-glycolide by the solvent evaporation method, which has a problem that, when the liquid phase is contained at a high concentration of 30% or more, the liquid phase is liable to volatilize during the preparation process, leading to difficulty in consistently encapsulating the fatty acid ester in the desired amount in the microspheres. This means that the encapsulation efficiency of cyclosporin, which is dissolved in the fatty acid ester, may be affected and there may be difficulty in obtaining microspheres of a uniform composition. Furthermore, as a relatively large amount of fatty acid esters are needed for achieving the release increase, this serves to be a further limiting factor in encapsulating cyclosporin in biodegradable polymer microspheres.

According to the results of the study, the amount of cyclosporin which can be encapsulated in practice is less than 20%. Considering that the dose of cyclosporin is relatively large, the fact that the amount of drug that may be encapsulated in any one dosage unit, clearly suggests that there will be difficulty in utilization as a sustained release preparation. The required daily dose of cyclosporin for a human patient is within the range of 60 mg/60 kg to 120 mg/60 kg. With the drug content being only 20%, the converted amount of cyclosporin-containing microspheres to last for one week, would require that 2.1 g to 4.2 g of microspheres would need to be administered, which would clearly result in patient compliance problems. Moreover, the volume of microspheres required, would be prohibitive in formulating an injectable formulation. Also, because fatty acid esters, of which pharmaceutical acceptability has not yet been established, would be contained in the formulation in a large amount, the possibility of inducing adverse effects, e.g., topical irritation and necrosis, cannot be completely excluded.

### SUMMARY OF THE INVENTION

In light of all the foregoing, the present inventors set out to develop a cyclosporin preparation based on new concept, one that minimizes adverse effects, that reduces medical expenses incurred for preliminary monitoring, that improves patient compliance, and that establishes a reliable drug administration regimen. Thus, it is an object of the present invention to provide an injectable cyclosporin preparation, particularly a cyclosporin-containing sustained-released pharmaceutical composition, that is capable of regulating and maintaining the blood concentration of the drug in the effective range for several days to several weeks by continuously releasing the drug over this period of time.

These objectives, as well as other features and advantages of the principals of the present invention will become readily apparent to the person of skill in the art after a thorough reading of the following detailed description when taken in conjunction with the accompanying drawings.

### BRIEF DESCRIPTION OF THE DRAWINGS

FIG. 1 is a scanning electron micrograph of microspheres prepared in accordance with the protocol set forth in Example 5;

FIGS. 2a and 2b show the results of the in-vitro release test of cyclosporin from microspheres of Comparative Example 1 (♦) and Examples 1 (Δ), 2 (▴), 3 (&Ovalsolid;), 4 ( [symbol] ) and 5 (&Rectversolid;), in which Tween 80 was added to the release medium (at a concentration of 0.025% in FIG. 2a and 0.05% in FIG. 2b) and the test tube was positioned perpendicular to a vibrating direction; and

FIG. 3 is the blood concentration-time profiles of cyclosporin following the subcutaneous injections of microspheres of Comparative Example 1 (♦) and Examples 3 (&Ovalsolid;) and 5 (&Rectversolid;) to Spraque-Dawley (“SD”) rats.

### DETAILED DESCRIPTION OF THE INVENTION

As used herein, the term “cyclosporin” refers to cyclosporin A and analogues of Cyclosporin A having similar physical properties.

The present invention relates to a cyclosporin-containing sustained release pharmaceutical composition. More particularly, the present invention provides a cyclosporin-containing sustained release pharmaceutical composition comprising cyclosporin and a release modifier encapsulated in a biodegradable polymer. Preferably, the release modifier is selected from the group consisting of hydrophilic release modifiers and lipophilic release modifiers, and combinations thereof.

In various embodiments, the composition may be in the form of microspheres or nanospheres.

In the pharmaceutical composition of the present invention, the amounts of cyclosporin, the biodegradable polymer and the release modifier are preferably 15 to 70%, 25 to 80% and 0.01 to 20%, and more preferably 25 to 60%, 35 to 70% and 0.1 to 10%, respectively.

The biodegradable polymer used in the composition of the present invention may be any injectable or implantable biodegradable polymer, and will preferably be selected from the group consisting of hydroxy acids such as polylactide (PLA) and polyglycolide (PGA); poly(lactide-co-glycolide) (PLGA), poly β-hydroxy butyric acid (PHB), polycaprolactone, polyanhydride, polyorthoester, polyurethane, poly(butyric acid), poly(valeric acid) and poly(lactide-co-caprolactone), as well as derivatives, copolymers and mixtures thereof.

The present inventors have discovered that the rate of drug release in vivo upon injection may be regulated by using the release modifier to prevent an interaction between cyclosporin and the biodegradable polymer, thereby promoting drug release from the biodegradable polymer.

The release modifier used in the composition of the present invention will preferably be selected from hydrophilic release modifiers and lipophilic release modifiers, and more preferably, a hydrophilic release modifier and a lipophilic release modifier are combined to ensure that the drug can be continuously released at a constant rate in vivo.

Hydrophilic release modifiers that can be used in the present invention include, for example, but are not limited to, polyoxyethylene sorbitan fatty acid esters, glyceryl monooleate, sorbitan fatty acid esters, poly(vinyl alcohol), poloxamers, poly(ethylene glycol), glyceryl palmitostearate, benzyl benzoate, ethyl oleate, α-cyclodextrin, β-cyclodextrin, γ-cyclodextrin, hydroxypropylβ-cyclodextrin and the like.

The hydrophilic release modifier contains such hydrophilic groups as hydroxy, ester, ethylene oxide, propylene oxide and the like, are pharmaceutically acceptable, and do not carry an electric charge. They induce an initial drug release by producing proper small pores inside of the microsphere at the early stage of drug release. Thus, they do not affect the solubility of cyclosporin, but form appropriate small pores in the structure of the microspheres, whereby amounts of the cyclosporin is withheld from an excessive initial drug release. The type and amount of the hydrophilic release modifier used to induce the initial release can vary depending on the kinds of the biodegradable polymer and the lipophilic release modifier used.

Lipophilic release modifiers that are appropriate for use in the present invention include, but are not limited to, for example, pharmaceutically acceptable natural oils such as soybean oil, cotton seed oil, sesame oil, peanut oil, canola oil, corn oil, coconut oil, rapeseed oil, theobroma oil and the like. This component acts to continuously induce drug release at the later stages by reducing the hydrophobic interaction between cyclosporin and the biodegradable polymer, which is believed to be a main cause of release obstruction at the later stages. The natural oils function as a buffer between the cyclosporin and the hydrophobic biodegradable polymer, thereby inhibiting the obstruction of drug release due to an interaction between the two components. Also, these natural oils are harmless to the human body and are commonly used in the preparation of injectable formulations. The type and amount of the lipophilic release modifier can vary depending on the kind of biodegradable polymer and hydrophilic release modifier used.

The hydrophilic and the lipophilic release modifiers may be used alone or in combination of at least two thereof to effectively regulate the release of the encapsulated cyclosporin.

Compositions according to the present invention may be administered by injection or implantation. More specifically, injection would include, for example, subcutaneous injection, intramuscular injection and the like. Formulations might include, for example, injectable solutions, powders for reconstitution, and implant.

Compositions according to the present invention may further comprise excipients, stabilizers, pH modifiers, isotonic agents and the like, as needed in preparing any of the aforementioned formulations for practical application.

Compositions according to the present invention may be prepared by methods such as freeze-drying, evaporation drying, spray drying, vacuum drying and the like. The production of the microspheres containing cyclosporin according to the present invention can be performed by the method such as water in oil single emulsion solvent evaporation and solvent extraction using an appropriate mixer commonly used, or by spray drying. In order to prepare the composition of the present invention having a desired release-controlling effect, it is important to produce microspheres in a short time under relatively mild conditions.

Compositions according to the present invention will maintain an in vivo cyclosporin blood concentration of 100 to 500 ng/ml for 7 to 28 days through the sustained release of cyclosporin.

As is generally observed immediately after oral administration of standard cyclosporin preparations, compositions of the present invention do not show a temporary increase in the blood concentration of cyclosporin, but uniformly maintain a pharmaceutically effective concentration, thereby resulting in a decreased risk of drug toxicity. Also, because the compositions of the present invention do not show individual differences in absorption ratio, it is possible to predict blood concentration. As a result, it is possible to omit initial procedures for unnecessary drug administration to determine the dose of cyclosporin preparations and blood concentration assay for the therapeutic drug monitoring (TDM). In addition, as the compositions release the drug at a constant concentration for several days to several weeks, daily administration is not required, and patient compliance will be improved.

Release Test of Cyclosporin

The present inventors have confirmed that, in the in vitro release test for the cyclosporin-containing microsphere preparation, when the composition of the release medium was changed, the in vitro release pattern was also altered. With this result, considering that the target formulation of the present invention was not intended for oral administration (but rather for injection or implant), we have come to expect that the in vitro release patterns obtained by the conventional method might not reflect the in vivo release patterns for the formulations of the present invention. Therefore, we have established an in vitro release test method suitable for the compositions of the present invention. The test method involves screening of the candidate compositions by analyzing the in vitro release patterns of cyclosporin through administration of the formulation to SD rats; thus carrying out a blood concentration assay.

From the experiments with various release media to establish optimal releasing conditions of microspheres in vitro, the present inventors have found that a release medium with polysorbate 80 (“Tween® 80”), was the most effective. According to the recent report of AAPS PharmSciTech 2001:2(1) article 2, as the concentration of Tween® 80 was increased 20 times, cyclosporin solubility was increased 60 to 160 times through micellization by the Tween® 80. Thus, the release pattern may be modulated through the control of a solubilization of cyclosporin encapsulated in microspheres, by adjusting the concentration of Tween® 80 within the range of 0.025 to 0.1%, in the release medium of sodium phosphate buffered saline of pH 7.5 containing 0.01% sodium azide.

10 mg of freeze-dried microspheres with encapsulated cyclosporin were dispersed in sodium phosphate buffered saline of pH 7.5 containing 0.025 to 0.1% (W/V) Tween® 80 and 0.01% sodium azide, followed by being subjected to the release test in vitro. A test tube for measurement of the released amount was placed in a water bath vibrating in a fixed direction at 37° C. and, such that the test tube was positioned perpendicular or horizontal to the vibrating direction. In the apparatus for the release test, it was observed that placement of the test tube in a perpendicular or horizontal direction to the vibrating direction in the water bath resulted in different cyclosporin release profiles. Particularly, when the test tube was placed in a horizontal direction to the vibrating direction in the water bath, the microspheres in the tube did not settle down due to the rapid movement of medium, but remained in the form of separate particles. As a result, water channels can be formed relatively readily and cyclosporin encapsulated in the microspheres can be dissolved out rapidly through the water channels of the hydrophobic microspheres. Alternatively, when the test tube was placed in a perpendicular direction to the vibrating direction in the water bath, the microspheres settled down and agglomerated with each other by gravity, due to the weight of the microspheres, and the cyclosporin was found to be released slowly. This is believed to be the results from the fact that the agglomerated microspheres lying in the bottom of the test tube had difficulty in forming water channels inside of the microspheres. Moreover, it is also believed to be the results from the fact that cyclosporin should be released from such conglomerates.

In the present invention, in order to predict the in vivo release pattern of cyclosporin, a system simulating circumstances in vivo upon administration of the microspheres was established by varying the concentration of Tween® 80 in in vitro release medium between 0.025 and 0.1% while placing the test tube in a perpendicular direction to a vibrating direction in the water bath, and used for this study.

The principals of the present invention will now be described in detail according to the following. It is understood, however, that such examples are provided for illustration only, and the invention is not intended to limited by the examples.

### EXAMPLES 1-5 AND COMPARATIVE EXAMPLE 1

Preparation of Microspheres Using PLGA 5015 as Biodegradable Polymer (Solvent Evaporation Method)

Microspheres were prepared by solvent evaporation method using W/O single emulsion, according to the formulations given in Table 1 below.

TABLE 1
Formulations of microspheres using PLGA 5015 as a biodegradable polymer

| | Comparative Example 1 | Example 1 | Example 2 | Example 3 | Example 4 | Example 5 |
|---|---|---|---|---|---|---|
| | CyA-PLGA | RP5 | RP10 | RP2S2 | RP5S5 | RP10S10 |
| Cyclosporin | 160 mg | 160 mg | 160 mg | 160 mg | 160 mg | 160 mg |
| Poly(lactide-co-glycolide) | 240 mg | 220 mg | 200 mg | 224 mg | 200 mg | 160 mg |
| PLGA5015 | | | | | | |
| Poloxamer ® 188 | — | 20 mg | 40 mg | 8 mg | 20 mg | 40 mg |
| Sesame Oil | — | — | — | 8 mg | 20 mg | 40 mg |

In Comparative Example 1 and Examples 1 to 5, poly(lactide-co-glycolide) (PLGA) (PLGA5015, Wako Pure Chemical Industry, Japan) having a molecular weight of 15000 (lactic acid:glycolic acid=50:50) was used.

A stirring apparatus was designed by fixing a blade with a diameter of 45 mm at a height of 30 mm from the bottom in a cylindrical container with a diameter of 70 mm and a height of 105 mm, which had 3 partitions with a thickness of 10 mm mounted on the surface of the cylindrical wall at 120 degree intervals, and used for preparation of microspheres.

Cyclosporin, poly(lactide-co-glycolide), Poloxamer® 188 and sesame oil were weighed, separately, in the amounts shown in Table 1, and added to a lidded container of appropriate dimensions. 4 ml of dichloromethane was added to the container and the container was sealed tightly, followed by stirring to completely dissolve the contents to obtain an oily solution (Solution 1). 150 ml of aqueous solution (Solution 2) containing 0.3% polyvinyl alcohol and 0.3% Tween® 80 was added to the container for preparation of microspheres and then Solution 1 was added to the Solution 2 while being stirred at 1000 rpm, followed by stirring at 1000 rpm for 30 minutes to form an O/W emulsion. The resulting emulsion was stirred for one more hour at 300 rpm to solidify microspheres. The solidified microspheres were separated by filtering through a cellulose acetate membrane of 0.22 μm, washed three times with distilled water, and freeze-dried for 24 hours. Thus, the preparations of the microspheres of Comparative Example 1 and Examples 1 to 5 was completed. All the processes described above were performed on a clean bench, and the level of aseptic conditions was maintained as high as possible.

### EXAMPLES 6-10 AND COMPARATIVE EXAMPLE 2

Preparation of Microspheres Using PLGA 5015 as a Biodegradable Polymer (Sonication Method)

These examples were performed using the same Solutions 1 and 2 as in Examples 1 to 5. Solution 1 was added to Solution 2. The resulting suspension was promptly dispersed by sonication at 70 mW for 3 minutes and stirred at 700 rpm for 2 hours by a magnetic stirrer to solidify microspheres. The solidified microspheres were separated by filtering through a cellulose acetate membrane of 0.22 μm, washed three times with distilled water, and freeze-dried for 24 hours. All the processes described above were performed on a clean bench and aseptic conditions were maintained as much as possible.

### EXPERIMENTAL EXAMPLE 1

Scanning Electron Microscopy of Microspheres

FIG. 1 shows the result of the scanning electron microscopy of microspheres prepared from Example 5. It was confirmed that uniform microspheres having particle size of less than 30 μm could be conveniently prepared by the method according to the present invention, even when 20% of a release modifier was added.

### EXPERIMENTAL EXAMPLE 2

Encapsulation Efficiency of Cyclosporin in Microspheres

In this example, the inventors used the physicochemical properties of methanol, that is, it can dissolve cyclosporin well while can not dissolve the biodegradable polymeric carriers for cyclosporin such as poly(lactide-co-glycolide), poly(lactide), and the like. It is an efficient method in that it can conveniently and precisely measure an encapsulated amount of cyclosporin in microspheres with high encapsulation amount of cyclosporin.

10 mg of microspheres containing cyclosporin in a large proportion (30 to 60%) were dispersed in 50 ml of methanol. The dispersion was subjected to sonication for 1 hour so that encapsulated cyclosporin was fully and rapidly extracted. The extracted cyclosporin in methanol was measured by reverse-phase high pressure liquid chromatography at a detection wavelength of 215 nm. Also, in order to confirm that cyclosporin contained in the microspheres had been completely extracted, the biodegradable polymers transformed into gel were measured using nuclear magnetic resonance spectroscopy.

The encapsulation efficiencies of cyclosporin in the microspheres prepared in Comparative Example 1 and Examples 1 to 5 are shown in Table 2. It was found that at least 95% of the cyclosporin was completely encapsulated into the microspheres prepared in Comparative Example 1 and Examples 1 to 5. The encapsulation efficiency was calculated by the equation (n=3):

Encapsulation Efficiency (%)=(amount of cyclosporin in 10 mg microspheres/4 mg*)×100*4 mg−Theoretical loading amount of cyclosporin

TABLE 2
Encapsulation efficiency of microspheres

| | Comparative | | | | | |
|---|---|---|---|---|---|---|
| | Example 1 | Example 1 | Example 2 | Example 3 | Example 4 | Example 5 |
| | CyA-PLGA | RP5 | RP10 | RP2S2 | RP5S5 | RP10S10 |
| Encapsulation | 99% (±2) | 105% (±3) | 103% (±2) | 95% (±4) | 98% (±5) | 102% (±3) |
| Efficiency | | | | | | |

### EXPERIMENTAL EXAMPLE 3

In vitro Release Test of Drug from Microspheres Containing Cyclosporin

10 mg of freeze-dried cyclosporin-containing microspheres were dispersed in sodium phosphate buffer of pH 7.5 containing 0.025 to 0.1% (W/V) Tween® 80 and 0.01% sodium azide, followed by subjection to a release test in vitro. A test tube for measurement of the released amount was placed in a water bath vibrating in a fixed direction at 37° C. and, at right angles to the vibrating direction.

In order to measure the released amount of cyclosporin, the test tube was centrifuged at a speed of 3000 rpm for 15 minutes at fixed time intervals, 50 ml of supernatant was obtained and then fresh medium of an equal volume was added promptly to the test tube. Using the release medium obtained from the supernatant, the released amount and the stability of cyclosporin was measured by reverse-phase high pressure liquid chromatography with UV detector at a wavelength of 215 nm. The reverse-phase high pressure liquid chromatography system is described as follows: Waters 510 HPLC pump system was connected to Waters 484 UV detector, the temperature of the column was kept at 70° C. and the mobile phase was a mixed solution of acetonitrile and water (80:20). As a column, a Phenomenex Column-Luna, RP-18 (4.6×250 mm, particle size 5 (m, USA) was used.

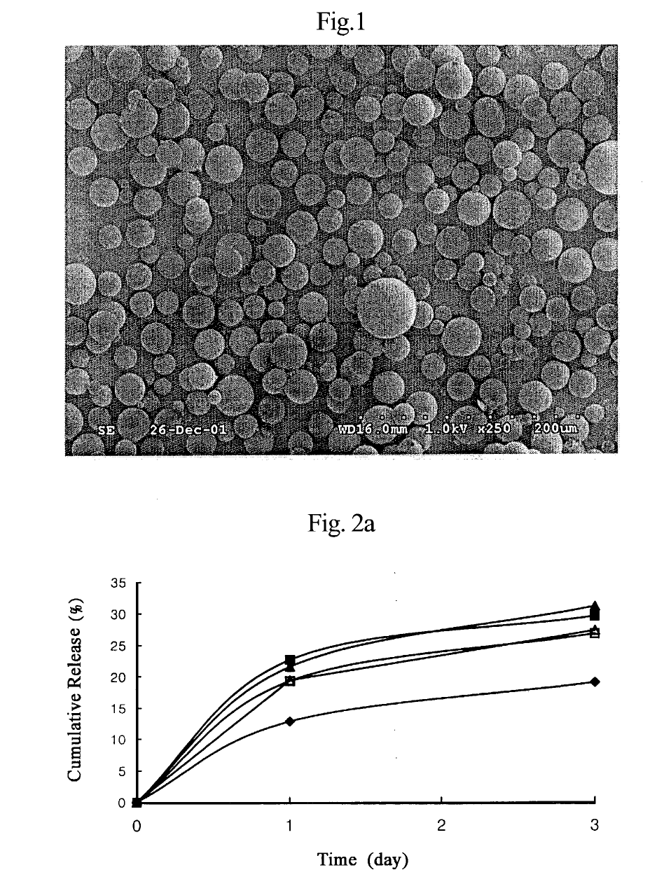

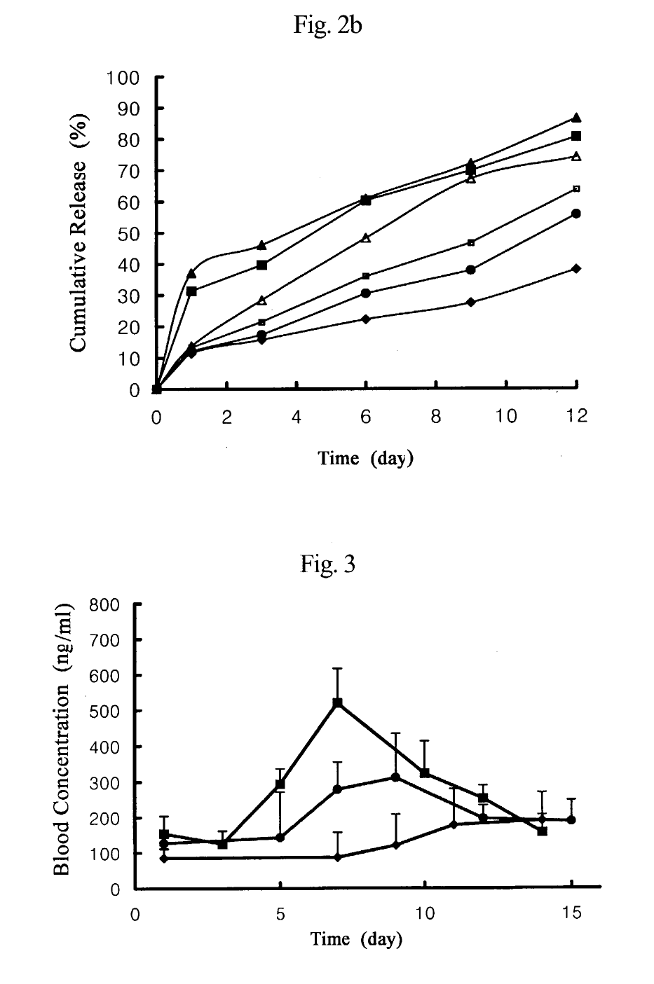

Upon examining the drug release patterns in vitro shown in FIGS. 2a and 2b, when the concentration of Tween® 80 was 0.025%, the compositions of Examples 1 to 5, which contain the release modifier, differed by about 15% in the amount of released cyclosporin from the composition of Comparative Example 1, which did not contain a release modifier, at the third day of test. However, it fails to show clearly the difference of release patterns depending on the content of the release modifier. Furthermore, it was not observed any increase of release amount of cyclosporin after the third day. On the other hand, when the concentration of Tween® 80 was increased to 0.05%, the difference of the drug release patterns depending on the content of the release modifier was shown to reach a maximum of 40% at the third day. In the present invention, the medium containing 0.05% Tween® 80 was selected as an in-vitro release medium for the use in the formulation screening test.

### EXPERIMENTAL EXAMPLE 4

In vivo Release Test of Drug From Microspheres Containing Cyclosporin

For in vivo drug release test, 200 g male Spraque-Dawley rats was subcutaneously injected with cyclosporin-containing microspheres suspended in a solvent for injection with amount of 37.5 mg/kg. The solvent for injection was 1.5% sodium carboxymethylcellulose solution in distilled water for injection containing 0.9% sodium chloride and 0.1% Tween® 20. Sodium chloride was used to make the injection solution isotonic for the alleviation of pain around the injection site. Sodium carboxymethylcellulose was used as a thickener to maintain the viscosity of the injection solution at 200 to 400 cps in order that microspheres can be effectively suspended in the solvent for injection, the injection solution can be maintained in the form of a homogeneous suspension during injection and the microspheres can be remained around the injection site after injection. Any thickener that is injectable and nontoxic can be employed, but the obtained injection solution is required to maintain the foregoing range of the viscosity. The solvent for injection was sterilized before use. Cyclosporin-containing microspheres were suspended at a concentration of 50 mg/ml just before use and then injected to SD rat in a converted amount on the basis of the weight of the rat. Here, a 22-gauge needle was used. The blood concentration of cyclosporin in the white mouse was determined by the cyclosporin monoclonal whole blood assay (TDx system, Abbott Lab., USA) with a fluorescence polarization immunoassay (FPIA) using whole blood.

As a consequence of the administration of cyclosporin-containing microspheres, it was shown that the blood concentration of cyclosporin varied considerably according to the content of the release modifier (FIG. 3). The group that did not contain a release modifier maintained a blood concentration of about 100 ng/ml, falling short of the effective blood concentration (Comparative Example 1 ♦). On the other hand, Examples 3 (&Ovalsolid;) and 5 (&Rectversolid;) that contained the release modifier according to the present invention appeared to maintain much higher blood concentration on the whole.

In addition, it was observed that Example 5 (&Rectversolid;), which contained Poloxamer® 188 and sesame oil as a release modifier in an amount of 10% separately, showed a maximum blood concentration of 500 ng/ml or higher, whereas Example 3 (RP2S2), in which the content of the release modifier was regulated to 2%, showed effective and constant blood concentration between 150 ng/ml to 350 ng/ml. These results indicate that the blood concentration can be controlled by adjusting the content of the release modifier. The type and amount of a release modifier can vary according to the type of a used biodegradable polymer and the cyclosporin content.

The sustained-release microspheres containing high concentration of cyclosporin, prepared according to the present invention, can release the whole quantity of cyclosporin encapsulated in microsphere at a constant rate while uniformly maintaining the therapeutically effective concentration of cyclosporin for several days to several weeks, which is required in cyclosporin preparations, and it is possible to minimize adverse effects that may occur due to non-uniform bioavailability caused by the oral administration, thereby accomplishing reduction of medical expenses incurred for a preliminary monitoring and improving patient compliance for medication.

We claim:

1. A pharmaceutical composition formulated for sustained release comprising cyclosporin and a release modifier encapsulated in a biodegradable polymer.

2. The pharmaceutical composition of claim 1, wherein the release modifier is selected from the group consisting of hydrophilic release modifiers, lipophilic release modifiers, and combinations thereof.

3. The pharmaceutical composition of claim 2, wherein the release modifier comprises at least one hydrophilic release modifier and at least one lipophilic release modifier.

4. The pharmaceutical composition of claim 2, wherein the release modifier comprises at least one hydrophilic release modifier selected from the group consisting of glyceryl monooleate, polyoxyethylene sorbitan fatty acid esters, sorbitan fatty acid esters, poly(vinyl alcohol), poloxamers, poly(ethylene glycol), glyceryl palmitostearate, benzyl benzoate, ethyl oleate, α-cyclodextrin, β-cyclodextrin, γ-cyclodextrin and hydroxypropyl β-cyclodextrin.

5. The pharmaceutical composition of claim 2, wherein the release modifier comprises at least one lipophilic release modifier selected from the group consisting of soybean oil, cottonseed oil, sesame oil, peanut oil, canola oil, corn oil, coconut oil, rapeseed oil and theobroma oil.

6. The pharmaceutical composition of claim 4, wherein the release modifier further comprises at least one lipophilic release modifier selected from the group consisting of soybean oil, cottonseed oil, sesame oil, peanut oil, canola oil, corn oil, coconut oil, rapeseed oil and theobroma oil.

7. The pharmaceutical composition of claim 1, wherein the biodegradable polymer is selected from the group consisting of polylactide and polyglycolide, poly(lactide-co-glycolide), poly β-hydroxy butyric acid, polycaprolactone, polyanhydride, polyorthoester, polyurethane, poly(butyric acid), poly(valeric acid), poly(lactide-co-caprolactone), and derivatives, copolymers and mixtures thereof.

8. The pharmaceutical composition of claim 1, wherein the biodegradable polymer, the cyclosporin and the release modifier form microspheres or nanospheres.

9. The pharmaceutical composition of claim 1, wherein the amounts of cyclosporin, biodegradable polymer and release modifier are respectively, 15 to 70%, 25 to 80% and 0.01 to 20%.

10. The pharmaceutical composition of claim 9, wherein the amounts of cyclosporin, biodegradable polymer and release modifier are respectively, 25 to 60%, 35 to 70% and 0.1 to 10%.

11. The pharmaceutical composition of claim 1, wherein the composition is formulated for injection.

12. The pharmaceutical composition of claim 11, wherein the composition is formulated for subcutaneous injection or intramuscular injection.

13. The pharmaceutical composition of claim 11, wherein the composition is formulated as an injectable solution or as a powder for reconstitution as an injectable solution.

14. The pharmaceutical composition of claim 1, wherein the composition is formulated for implant.

15. The pharmaceutical composition of claim 1, wherein the composition is formulated to release the cyclosporin over a sustained period such that upon administration to a patient in need thereof, a blood cyclosporin concentration of 100 to 500 ng/ml is maintained in vivo for 7 to 28 days.
